(19) United States
(12) Patent Application Publication
(10) Pub. No.: US 2024/0221880 A1
(43) Pub. Date: Jul. 4, 2024

(54) APPARATUS FOR PREVENTING DECLINE OF LONGEVITY

(71) Applicant: Oceandrive Ventures, LLC, Rio Grande, PR (US)

(72) Inventor: Jeffrey Gladden MD FACC, Rio Grande, PR (US)

(73) Assignee: Oceandrive Ventures, LLC, Rio Grande, PR (US)

(21) Appl. No.: 18/091,086

(22) Filed: Dec. 29, 2022

(51) Int. Cl. G16H 20/00; G16H 50/70

(52) U.S. Cl. CPC G16H 20/00; G16H 50/70

(57) ABSTRACT

An apparatus for preventing loss of longevity is disclosed. The apparatus may include at least a processor. The apparatus may include a memory communicatively connected to the at least a processor, the memory containing instructions configuring the at least a processor to receive a longevity parameter from a user, compare the longevity parameter to a decline threshold, identify a longevity decline driver as a function of the comparison, classify the longevity decline driver to a longevity decline stage and generate a longevity plan as a function of the longevity decline stage.

### FIELD OF THE INVENTION

The present invention generally relates to the field of medical treatments. In particular, the present invention is directed to an apparatus for preventing loss of longevity.

### BACKGROUND

Extending human longevity is a complex, multifaceted problem. Depending on health status, issues, and goal of patients, a personalized treatment may be necessary to help patients improve their overall health, and thus live longer. Existing solutions to this problem are not satisfactory.

### SUMMARY OF THE DISCLOSURE

In an aspect, an apparatus for preventing loss of longevity is disclosed. The apparatus may include at least a processor. The apparatus may include a memory communicatively connected to the at least a processor, the memory containing instructions configuring the at least a processor to receive a longevity measurement parameter from a user, identify a longevity parameter as a function of the longevity measurement, compare the longevity parameter to a decline threshold, identify longevity decline driver as a function of the comparison, determine classify the longevity decline driver to a longevity decline stage as a function of the longevity decline driver and generate a longevity plan as a function of the longevity decline stage.

In another aspect, a method for preventing loss of longevity. The method may include receiving, using a processor, a longevity parameter from a user. The method may include comparing, using the processor, the longevity parameter to a decline threshold. The method may include identifying, using the processor, a longevity decline driver as a function of the comparison. The method may include classifying, using the processor, the longevity decline driver to a longevity decline stage. The method may include generating, using the processor, a longevity plan as a function of the longevity decline stage.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for preventing loss of longevity;

FIG. 2 is a block diagram of an exemplary machine-learning process;

FIG. 3 is a block diagram of an exemplary embodiment of a longevity database;

FIG. 4 is a diagram of an exemplary embodiment of neural network;

FIG. 5 is a diagram of an exemplary embodiment of a node of a neural network;

FIG. 6 is a flow diagram of an exemplary method of preventing loss of longevity; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for preventing loss of longevity. In an embodiment, the processor may receive a longevity parameter from a user. Aspects of the present disclosure can be used to identify a longevity decline driver as a function of the longevity parameter. Aspects of the present disclosure can also be used to classify the longevity decline driver to a longevity decline stage. Aspects of the present disclosure allow for the processor to generate a longevity plan as a function of the longevity decline stage. Exemplary embodiments illustrating aspects of the present disclosure are described below in the context of several specific examples.

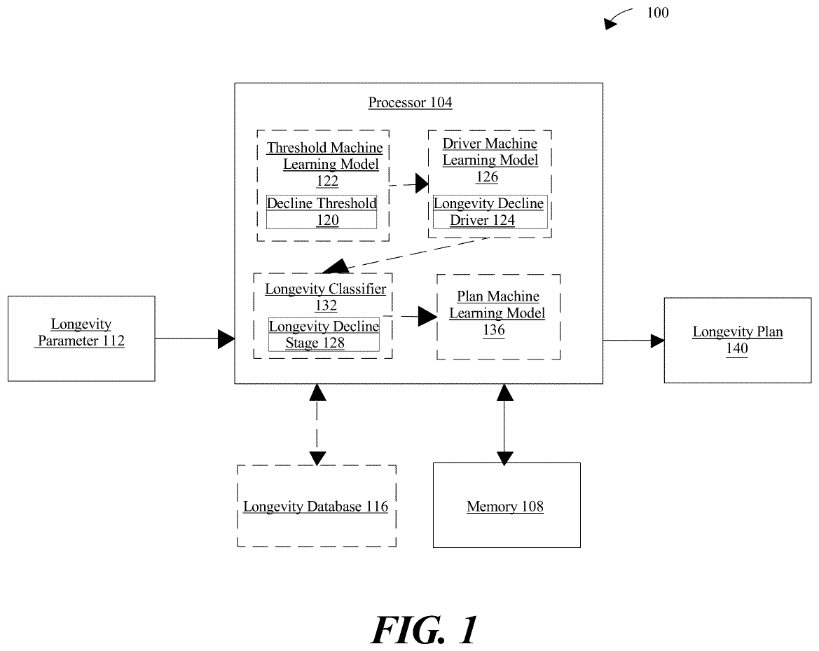

Referring now to FIG. 1, an exemplary embodiment of apparatus 100 for preventing decline of longevity is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 contains a memory 108 communicatively connected to at least a processor 104. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

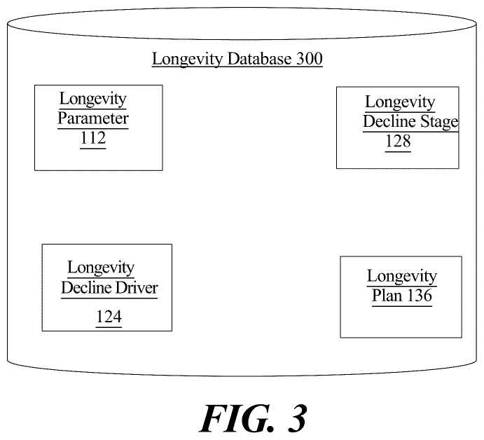

With continued reference to FIG. 1, memory 108 contains instructions configuring processor 104 to receive longevity parameter 112. As used in this disclosure, a “longevity parameter” is data that relates to the age of the systems of a user in different categories. The user disclosed herein is described below. In some embodiments, the categories of a user's systems may include, without limitation, life energy, health, longevity, performance, and the like thereof. In some embodiments, longevity parameter 112 may be stored in a longevity database 116, such as longevity database 300 disclosed with reference to FIG. 3. As used in the current disclosure, “systems” are a human's biological systems. A user's systems may include but are not limited to the circulatory, nervous, skeletal, respiratory, reproductive, endocrine, integumentary, renal, digestive, and muscular systems. Each organ may have one or more specialized roles in the body and is made up of distinct tissues. Additionally, a user's systems may include the heart, lungs, kidneys, or any other organ system. This may also include the components of a given system. In a non-limiting example, the lungs may be component of the respiratory system. In some embodiments, longevity parameter 112 may take into account chemical, biological, physical, and behavioral data relating to the user. Additionally, longevity parameter 112 may also evaluate a user's age as it relates to their natural, social, and built environments. As a non-limiting example, longevity parameter 112 may be a combination of a user's health data including medical history, user diet, exercise, sleep, data corresponding to timestamps and geographical locations for how a user spends his or her time, data regarding how the user spends money, user social media information, and the like. In other embodiments, longevity parameter 112 may include information collected from a standard health screening such as electrolyte level, kidney function, blood count, hydration status, and the like. As a non-limiting example, the health screening may include tests like Complete Blood Count, Prothrombin Time. Basic Metabolic Panel, Comprehensive Metabolic Panel, Lipid Panel. Liver Panel, Thyroid Stimulating Hormone, Hemoglobin AlC. In some embodiments, the health measurement may include more comprehensive analysis such as results from a screening test or diagnostic medical procedure.

With continued reference to FIG. 1, processor 104 is configured to receive longevity parameter 112 from a user. As used in this disclosure, “receive” from a user means accepting, collecting, or otherwise receiving input from a user and/or device. As used in this disclosure, an “user” is an individual who interfaces with apparatus 100. For example, the user may be a patient. As another example, the user may be a doctor. In some embodiments, longevity parameter 112 may include a user's anatomy data as it relates to age. As used in this disclosure, “anatomy data” is any data indicative of a user's physical health as it relates to age. In some embodiments, the user's physical health may include the health of various user systems including the user's circulatory system, a digestive system, a nervous system, reproductive system, endocrine system, skeletal system, and/or the like. This may additionally include one or more organs within each system within a user's body. Additionally, anatomy data may include various information about the users cell, tissues, bodily fluid, and the like. A user's anatomy data may be gathered by using a plurality of plurality of tests. As used in the current disclosures, “tests” refers to any medical test used to extract information about a user's systems. Tests may include various blood, imaging, functionality, lab tests, sleep tests, psychological evaluations, blood tests, urine tests, stool samples, evaluations by a medical professional, and the like. Examples of tests may include various blood tests like complete blood count test, a basic metabolic panel, a blood enzyme test, cholesterol tests, triglyceride tests, blood clotting tests, blood glucose test, blood oxygen test, and the like. Additional tests may be used to generate anatomy data may include various imaging tests such as an MRI, Xray, Mammogram, ultrasound, fluoroscopy, pet scans, and the like. A person who is reasonably skilled in the art, after having reviewed the entirety of this disclosure, would appreciate that various types of tests may be used determine the age, health, or status of the user's system.

With continued reference to FIG. 1, in some embodiments, longevity parameter 112 may include information from a user questionnaire, graphical user interface (GUI), or any other suitable forum for gathering information regarding the user's longevity. As used in this disclosure, “questionnaire” is a set of questions in the form of one or more data fields requesting that the user identify activities in which the user engaged. In an embodiment, questions presented to a user may include a number of times that a user engages in physical activity during a given period of time such as a day, a week or a year. In another embodiment, questions may also be directed to the substance abuse and the severity of the said substance abuse. This may include questions about how much and how often a user's drinks alcohol or does illegal substances. A user's responses may be verified by an evaluation by a medical professional to ensure accuracy. A medical professional may look for overt signs of activities that may affect a user's health. For example, a user may indicate that they rarely smoke cigarettes, however upon physical examination of a user's lungs it is apparent that the user is a heavy smoker. Persons skilled in the art, upon review of this disclosure in its entirety, will be aware of the various ways in which longevity measurement may be collected and provided to the system described herein. Further, in some cases, the longevity measurement may be stored in longevity database 116. In some cases, the longevity measurement may be retrieved from longevity database 116.

With continued reference to FIG. 1, in some embodiments, longevity parameter 112 may include one or more measurable longevity markers. As used in this disclosure, a “longevity marker” is a biomarker that represent one or more aspects of the longevity of the user. As used in the current disclosure, a “biomarker” is a biological molecule found in blood, other body fluids, or tissues that is a sign of a normal or abnormal process, or of a condition or disease. A biomarker may be used to see how well the body responds to a treatment for a disease or condition. A measurable biomarker may include, but is not limited to, yH2A.X immunohistochemistry, Leukocyte telomere length, MIR31HG, p16INK4a, Senescence-associated secretory phenotype (SASP) proteins, Measures of DNA methylation, SIRT1, SIRT2, SIRT3, SIRT6, SIRT7, Dosage of circulating microRNAs (miR-34a, MiR-21, miR-126-3p, miR-151a-3p, miR-181a-5p, miR-1248), P31 MRI spectroscopy, growth differentiating factor 15 (GDF15), Target of rapamycin (TOR), Protein carbonylation, Advanced glycation end products, Insulin-like growth factor (IGF-1), HGBA1c, IL-6, TNF-α, CRP (C-reactive protein), and TNFRII (tumor necrosis factor-α RII). These biomarkers may be measured using various pathways including, but is not limited to, DNA repair mechanisms, DNA modifications, telomere length, markers of DNA damage response, telomerase activity, senescent markers in blood and tissue, DNA methylation, histone acetylation, noncoding RNA, autophagy markers, chaperon proteins, proliferative capacity in vitro, growth hormone axis, and metabolism alterations. In an embodiment, the longevity markers may include, but it is not limited to, telomerase length/age, mitochondrial function, DNA repair, function of STEM cells, epigenetic rate of aging, senescent cell burden, immune system function, inflammation, mTOR AMPK balance, epigenetic urine, senescent cell SASP, and the like.

With continued reference to FIG. 1, in some embodiments, longevity parameter 112 may include longevity measurement. A “longevity measurement,” as used in this disclosure, is a measurement of user's systems, life energy, longevity age, health age, or performance metrics. In some embodiments, the longevity measurement may include a life energy measurement. A “life energy measurement”, as used in this disclosure, is a numeric measurement used to represent, measure, calculate, monitor, experiment, and/or evaluate the amount or level of the life energy flowing inside the energy meridian of a human or life body. As used in the current disclosure, “Life energy” is a form of energy related to but is not limited to, the brain, lungs, liver, bladder, kidneys, heart, stomach, and intestines in the human body. The Life energy measurement may also include an evaluation of the mental health status of human or life. This may include an evaluation of the mental health of human or life regarding overall energy, safety, wisdom, spiritual health, overall joy, relationship health, growth mindset, and overall mental health. In some cases, life energy measurement may be measured and/or derived by evaluation of telomeres, mitochondria, proteomic measures and/or evaluations, glycans measures and/or evaluations, oncogenic measures and/or evaluations, determinations of epigenetic rates of aging, epigenetic urine measures and/or evaluations, DNA repair measures and/or evaluations, epigenetic intrinsic measures and/or evaluations, epigenetic extrinsic measures and/or evaluations, mTor AMPK balance measures and/or evaluations, NAD measures and/or evaluations, NADH measures and/or evaluations, determinations regarding prevalence, health or other qualities of stem cells, senescent cell burden measures and/or evaluations, senescent cell SASP measures and/or evaluations, immune system measures and/or evaluations, and/or inflammation measures and/or evaluations. In an embodiment, the calculation of the life energy measurement may encompass an evaluation from a mental health professional and user inputs. In another embodiment, the calculation of the life energy measurement may include a life energy comparison metric. A “life energy comparison metric,” as used in this disclosure, is a comparison of the user's current state of the life energy compared to the average or/and healthy state of the life energy of one or more similar situated (age, gender, weight, height, and the like) users.

With continued reference to FIG. 1, in some embodiments, a longevity measurement may include a health age related to the user. As used in this disclosure, a “health age” is a comprehensive measurement related to the overall health status of the user. The health age may be generated as a function of the life energy measurement of the user. In a non-limiting example, a 50-year-old user with a high life energy measurement, wherein the high life energy measurement may be equivalent to a 25 years old. In an embodiment, the health age may include, but is not limited to, the evaluation of the gender, weight, substance abuse, family health history, life energy, overall health, performance metrics and the like. In other embodiment, the health age may be measured using one or more patient specific metrics. A “patient specific metric” is specific performance metric based on the current physical or mental health condition of the user. The patient specific metric may include, but is not limited to, current sleep status, vascular and anatomic function, lung health, sex hormone balance, sexual function, brain function, brain anatomy, bone health, muscle function, joint function, ligament function, and tendon function. In other embodiment, the health age may also include evaluation of one or more lifestyle choices of a user. As used in this disclosure, a “lifestyle choice” is a personal and conscious decision to perform a behavior that may increase or decrease the risk of injury or disease, or/and the overall quality of life of a human being. In a non-limiting example, a healthy lifestyle choice can benefit both physically and mentally in everyday life of the user. The lifestyle choice may include, but is not limited to, exercising regularly, maintaining a healthy body weight, avoiding stationary sitting, avoiding sugar, eating more vegetables and fruits, drinking more water, going to bed early, quitting smoking and the like. In an embodiment, calculation of the health age may include a health age comparison metric. A “health age comparison metric,” as used in this disclosure, is a comparison of the user's current overall health status compared to the average or/and healthy overall health status of one or more similarly situated (age, gender, weight, height, and the like) users.

With continued reference to FIG. 1, in some embodiments, a longevity measurement may include a longevity age related to the user. As used in this disclosure, a “longevity age” is a biological measurement related to the user representing the length or the duration of maintaining user's health age. In an embodiment, a longevity age may be the time from the users current age until system failure and/or death. The longevity age may be calculated as a function of the health age and the life energy measurement of the user. The longevity age may be determined by a rate of aging of the user. As used in this disclosure, a “rate of aging” is an indication of the rate of change of age as a function of the current status of a given system. Rate of aging may be used to determine how quickly a given system is aging. Rate of aging may also apply to a given component of a system. Rate of aging may be reflected in terms of a ratio or a fraction. In a non-limiting example, a user with a high rate of aging may have a lower longevity age, because the user may be aging quickly. In another non-limiting example, a user with a low rate of aging may have a higher longevity age, because the user may be aging slowly. The difference between a first and a second longevity parameter as numerator and the time between evaluations as denominator. Wherein the first longevity parameter (CLP₁) is taken at the first evaluation and the second longevity parameter (CLP₂) is taken at the second evaluation.

$$Rate of Aging = \frac{{CLP}_{1} - {CLP}_{2}}{Time between evaluations}$$

For example, a 25-year-old user's overall health status is similar to a 35-year-old on the first evaluation, therefore the user may have a longevity parameter of 10. During the second evaluation, approximately 1 year later, the 25-year-old user may have an overall health status similar to 40-year-old, therefore, the user may have a longevity parameter of 15. The rate of aging of the 25-year-old user will be approximately −5. This may mean that the user's systems are aging at 5× the rate of a normal person. In reference to the rate of aging, a value ranging from −1 to 1 may be considered healthy. In an embodiment, the further the rate of aging is away from 0, the faster the user is aging either positively or negatively. Positive ageing is when the system is getting progressively healthier and thus more comparable to a younger person. Negative aging is when the system is getting progressively older and thus more comparable to an older person.

With continued reference on FIG. 1, in some embodiments, a longevity measurement may include a performance age related to the user. As used in this disclosure, a “performance age” is a biological measurement that measures the user's ability to perform one or more predetermined tasks. Abilities that may be tested in evaluating a performance age of a user may include but not limited to the user's speed, flow state, agility, resilience, strength, reflection, both static and dynamic balance, recovery, flexibility, endurance, and the like. In an embodiment, the performance age may be generated as a function of the longevity age. In a non-limiting example, a user with a high longevity age may have a high-performance age, since the user may have a longer duration of maintaining a health age. In another non-limiting example, a user with a low longevity age may have a low performance age, since the user may have a shorter duration of maintaining a health age. In another embodiment, performance age may include a performance age comparison metric. A “performance age comparison metric,” as used in this disclosure, is a comparison of the user's current ability to perform one or more tasks compared to the average or/and expected ability to perform the same tasks of one or more similar situated (age, gender, weight, height, and the like) users.

With continued reference to FIG. 1, in some embodiments, at least a processor 104 may receive longevity parameter 112 from wearable device data that tracks how a user relates with his or her environments. As used in the current disclosure, a “wearable device” is a computing device that is designed to be worn on a user's body or clothing. The wearable device may detect wearable device data. In embodiments, a wearable device may include a smart watch, smart ring, fitness tracking device, and the like. As used in the current disclosure, “wearable device data” is data collected by a wearable device. Wearable device data may include data and associated analysis corresponding to, for instance and without limitation, accelerometer data, pedometer data, gyroscope data, electrocardiogramata, electrooculography (EOG) data, bioimpedance data, blood pressure and heart rate monitoring, oxygenation data, biosensors, fitness trackers, force monitors, motion sensors, video and voice capture data, social media platform data, and the like. In some embodiments, longevity parameter 112 may be provided by a user or a second individual on behalf of a user, for instance and without limitation a physician, medical professional, nurse, hospice care worker, mental health professional, and the like. Additionally without limitation, more descriptions on longevity parameter 112 and a longevity measurement may be found in U.S. patent application Ser. No. 18/073,064, filed on Dec. 1, 2022, and entitled “AN APPARATUS FOR ENHANCING LONGEVITY AND METHOD FOR ITS USE,” the entirety of which is incorporated by reference herein in its entirety.

With continued reference to FIG. 1, processor 104 is configured to compare longevity parameter 112 to decline threshold 120. As used in this disclosure, a “decline threshold” is a value related to a user's longevity that is set as a standard to determine a decline of longevity. As used in this disclosure, a “decline of longevity” refers to loss of longevity. As a non-limiting example, longevity parameter 112 of grip strength may be compared to decline threshold 120 of 50 kg for grip strength. As another non-limiting example, there may be decline threshold 120 for a performance age. In an embodiment, decline threshold 120 may indicate a balance point of longevity parameter 112 of the user. In another embodiment, decline threshold 120 may include one or more values and/or ranges of values relating to genomics, nutrigenomics, transcriptomics, proteomics, metabolomics, and the like. In some cases, decline threshold 120 may include a plurality of decline threshold 120. In other embodiments, decline threshold 120 may include one or more longevity parameter 112 of similar situated (age, gender, weight, height, and the like) users. As a non-limiting example, decline threshold 120 for a user who drinks alcohol may include longevity parameter 112 of another user with same age, gender and weight but who is a non-drinker. In some embodiments, decline threshold 120 may include a historical longevity parameter of the users, wherein the historical longevity parameter is any longevity parameter that is obtained any time before present time. As a non-limiting example, the historical longevity parameter may include a patient's a grip strength value that is measured in a previous visit to a health center. By comparing longevity parameter 112 with the historical longevity parameter may allow to see the health status of a user over time. In some embodiments, decline threshold 120 may be manually set by a user. As a non-limiting example, decline threshold 120 may be manually set by a medical professional. In some embodiments, decline threshold 120 may be stored in longevity database 116. In some cases, decline threshold 120 may be retrieved from longevity database 116. As a non-limiting example, previously used decline threshold 120 may be retrieved from longevity database 116. Additionally without limitation, more description of decline threshold 120 and the use of decline threshold 120 may be found in U.S. patent application Ser. No. 18/073,022, filed on Dec. 1, 2022, and entitled “AN APPARATUS FOR ENHANCING LONGEVITY AND METHOD FOR ITS USE,” the entirety of which is incorporated by reference herein in its entirety.

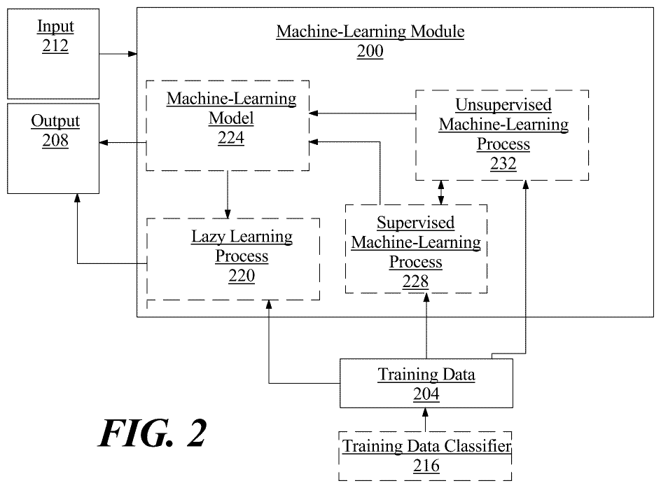

With continued reference to FIG. 1, in some embodiments, processor 104 may generate a decline threshold 120 using a threshold machine-learning model 122. For the purposes of this disclosure, a “threshold machine-learning model” is a machine-learning model that is configured to calculate a decline threshold. In some embodiments, training data of a threshold machine-learning model 122 may include longevity parameter 112 correlated to outputs that may include decline threshold 120. As a non-limiting example, inputs of a threshold machine-learning model 122 may include performance age, longevity age, heath age, life energy measurement, longevity markers, information from a user questionnaire and/or a standard health screening, anatomy data, and the like. As a non-limiting example, outputs of a threshold machine-learning model 122 may include a decline threshold 120 of performance age of 50 year-old. As a non-limiting example, outputs of a threshold machine-learning model 122 may include a decline threshold 132 of information from a user questionnaire of a number of time a user smoke in a week. Additionally, the threshold machine learning model 122 may be consistent with a machine learning module disclosed with respect to FIG. 2. In some embodiments, processor 104 may generate decline threshold 120 using a lookup table. A “lookup table,” as used in this disclosure, is an array of data that maps input values to output values. A lookup table may be used to replace a runtime computation with an array indexing operation. As a non-limiting example, longevity parameter 112 of 50 year-old man's breathing function test performance age may “lookup” decline threshold 120 of breathing function test performance age of similar situated user.

With continued reference to FIG. 1, processor 104 may be configured to identify longevity decline driver 124 as a function of a comparison of longevity parameter 112 and decline threshold 120. As used in this disclosure, a “longevity decline driver” is a factor that affects the decline in length and/or quality of the life span of a user. In some embodiments, longevity decline driver 124 may indicate cancer risk, musculoskeletal health, organ health, gut health, brain health, peripheral neuropathy, long haul covid, heart health, lung health, kidney health, liver health, pancreatic health, cosmetics, altered cell communication, genomic instability, telomere attrition, epigenetic alterations, loss of proteostasis, deregulated nutrient-sensing, mitochondrial dysfunction, cellular senescence, stem cell exhaustion, and the like. In an embodiment, longevity decline driver 124 may include longevity parameter 112 that is above decline threshold 120. As a non-limiting example, when a number of times a user smoke in a week is above decline threshold 120 of 5 times, the longevity parameter 112 may be longevity decline driver 124. In another embodiment, longevity decline driver 124 may include longevity parameter 112 that is below decline threshold 120. As a non-limiting example, when a grip strength performance metric is lower than decline threshold 120 of 50 kg, the longevity parameter 112 may be longevity decline driver 124. In some embodiments, longevity decline driver 124 may be stored in longevity database 116. In some embodiments, longevity decline driver 124 may be retrieved from longevity database 116. In some embodiments, longevity decline driver 124 may include a problematic area of the user. As used in this disclosure, a “problematic area” is an area associated with longevity parameter 112 that is below decline threshold 120. In an embodiment, problematic area of the user may be an aspect where user lack essential elements or/and need to be improved. In another embodiment, the problematic area may be an unhealthy system of the user. For example, an area x of the user that is currently evaluated through the longevity parameter 112 (LP_{current}), with measurements that are discovered to be less than the decline threshold 120 (θᵢ) is determined as a problematic area.

$$∀ x ∈ {LP}_{current} : x < {θ}_{1}$$

Additionally without limitation, the more description of problematic area may be found in U.S. patent application Ser. No. 18/073,064, filed on Dec. 1, 2022, and entitled “AN APPARATUS FOR ENHANCING LONGEVITY AND METHOD FOR ITS USE,” the entirety of which is incorporated by reference herein in its entirety.

With continued reference to FIG. 1, longevity decline driver 124 may include a driver weight. As used in this disclosure, a “driver weight” is a statistical weight assigned to a comparison of a longevity parameter and a decline threshold indicating the importance level of variables that drives a decline in longevity of a user. In an embodiment, evaluation score may be used to determine the degree of importance of longevity decline driver 124. In some embodiments, the driver weight may include a range of numbers or grades. As a non-limiting example, longevity decline driver 124 with higher driver weight may induce a decline of longevity more than longevity decline driver 124 with lower driver weight. In a non-limited example, the driver weight of longevity decline driver 124 of grip strength performance metric may be scored from 0 to 100. The driver weight of grip strength performance metric that is close to 0 may indicate the importance level of the grip strength performance metric causing decline of longevity is low. The driver weight of the grip strength performance metric that is close to 100 may mean the importance level of the grip strength performance metric causing decline of longevity is high. As another non-limiting example, the driver weight of longevity decline driver 124 of the number of times a user smokes in a week may be scored from 0 to 100. The driver weight of the number of times the user smokes in a week that is close to 0 may indicate the importance level of the number of times the user smokes in a week is low. The driver weight of the number of times the user smokes in a week that is close to 100 may indicate the importance level of the number of times the user smokes in a week is high. In some embodiments, the driver weight may be calculated as a function of the comparison of the longevity parameter 112 to the decline threshold 120 and divide the resulting value to the average of other users. As a non-limiting example, longevity parameter 112, such as not limited to performance age of 60-years old, that falls below decline threshold 120, such as not limited to 40-years old, by 20 years may include the driver weight of 4 that is calculated by dividing 20 by an average of performance age difference of other users, such as not limited to 5 years. In some embodiments, the driver weight may be proportional to the difference between longevity parameter 112 and decline threshold 120. As a non-limiting example, 20 years performance age difference calculated by comparing longevity loss parameter 112 such as not limited to performance age of 60-years old and decline threshold 120 such as not limited to 40-years old may include bigger driver weight than 10 years performance age difference calculated by comparing longevity loss parameter 112 such as not limited to performance age of 50-years old and decline threshold 120 such as not limited to 40-years old.

With continued reference to FIG. 1, in some embodiments, processor 104 may be configured to identify a plurality of longevity decline drivers 124. In some embodiments, the plurality of longevity decline driver 124 may include a plurality of driver weights. In some embodiments, processor 104 may compare a plurality of driver weights with each other. As a non-limiting example, the driver weight of the grip strength performance metric may be compared with the number of times a user smokes in a week. As a non-limiting example, the driver weight of 30 for the grip strength performance metric may be considered low when it gets compared with the driver weight of 80 for the number of times a user smokes in a week. In some embodiments, processor 104 may be configured to determine a core longevity decline driver within the plurality of longevity decline drivers 124. As used in this disclosure, a “core longevity decline driver” is a longevity decline driver that affects the life span of a user the most. In some embodiments, the core longevity decline driver may be determined as the function of its driver weight. For example, longevity decline driver 124 with the highest driver weight may be selected as the core longevity decline driver. As a non-limiting example, longevity decline driver 124 with the driver weight of 80 may be determined as the core longevity decline driver among longevity decline driver with driver weight of 10, 20, 44, 60, 75. Additionally without limitation, the driver weight disclosed herein may be consistent with an assigned weight disclosed in U.S. patent application Ser. No. 18/073,064, filed on Dec. 1, 2022, and entitled “AN APPARATUS FOR ENHANCING LONGEVITY AND METHOD FOR ITS USE,” the entirety of which is incorporated by reference herein in its entirety. Additionally without limitation, the driver weight disclosed herein may be consistent with a longevity factor disclosed in U.S. patent application Ser. No. 18/072,987, filed on Dec. 1, 2022, and entitled “AN APPARATUS FOR EXTENDING LONGEVITY AND METHOD FOR ITS USE,” the entirety of which is incorporated by reference herein in its entirety.

With continued reference to FIG. 1, in some embodiments, longevity decline driver 124 and/or driver weight may be determined using a driver machine learning model 126. As used in this disclosure, a “driver machine learning model 126” is a machine learning model that determines a longevity decline driver. In some embodiments, the driver machine learning model 126 may be consistent with machine learning module described with respect to FIG. 2. In some embodiments, the driver machine learning model 126 may be trained with driver training data, wherein the driver training data may contain a plurality of inputs containing a comparison of longevity parameter 112 and decline threshold 120 correlated to a plurality of outputs containing longevity decline driver 124 and/or driver weight. As a non-limiting example, the plurality of inputs may include difference between 50 kg grip strength threshold and 40 kg grip strength longevity parameter 112, wherein the longevity decline driver 124 may include driver weight of 10. In some embodiments, machine-learning process may generate a function of the trained machine-learning process to determine longevity decline driver 124 and/or driver weight.

With continued reference to FIG. 1, processor 104 is configured to determine a longevity decline stage 128 as a function of a longevity decline driver 124. In some embodiments, processor 104 may determine longevity decline stage 128 as a function of a driver weight. As a non-limiting example, As used in this disclosure, a “longevity decline stage” is a stage of a decline of longevity. In some embodiments, longevity decline stage 128 may be stored in longevity database 116. In some embodiments, longevity decline stage 128 may be retrieved from longevity database 116. In some embodiments, longevity decline stage 128 may include a first stage. As used in this disclosure, a “first stage” is a stage where decline of longevity is self-preventable. For example, longevity decline driver 124 of the first stage may include high number of times of smoking, high number of times of drinking, low number of times of exercising, high exposure to radiation EMF and pollutants, mild skin infections, mild trauma, poor lifestyle choices, unavailability to take a variety of nutrient dense foods, lack of exposure to sunshine, inadequate sleep habit, low growth mindset, a less sense of life purpose, high stress, and the like thereof.

With continued reference to FIG. 1, in some embodiments, longevity decline stage 128 may include a second stage As used in this disclosure, a “second stage” is a stage where decline of longevity requires professional intervention. For example, longevity decline drivers 124 of the second stage may include genomic instability, loss of proteostasis, compromised autophagy, microbiome disturbance, inflammation/oxidative stress, epigenetic alterations, deregulated nutrient sensing: NAD/NADH SIRTs, GH/IGF-1/ATP and nutrient depletion mTOR/AMPK, insulin/glucose, immuno-senescence, oncogenic potential, oncogenic potential mitigation, hormone and thyroid depletion, telomere attrition, stem cell exhaustion, cellular senescence and SASP, mitochondrial dysfunction, altered intracellular communication, and the like thereof.

With continued reference to FIG. 1, in some embodiments, longevity decline stage 128 may include a third stage. As used in this disclosure, a “third stage” is a stage wherein an ability of the user to return to a health state is precluded. As used in this disclosure, a “health state” is a state of a user's physical, mental and social well-being in good health. As a non-limiting example, the third stage of longevity decline stage 128 may include a serious liver infection caused by hepatitis D, defective autophagy, and the like.

With continued reference to FIG. 1, processor 104 may be designed and configured to create a machine-learning model using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm 16 mounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

With continued reference to FIG. 1, processor 104 may be configured to use longevity classifier 132 to classify longevity decline driver 124 to longevity decline stage 128. For example, processor 104 may take inputs of longevity decline driver 124 with a driver weight and sort into longevity decline stage 128, such as a first stage, a second stage and a third stage.

In some embodiments, processor 104 may be configured to generate longevity classifier 132 using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)-P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, processor 104 may be configured to generate longevity classifier 132 using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements. With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number experience of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values. Additionally without limitation, more descriptions of longevity classifier 132 disclosed herein may be found in U.S. patent application Ser. No. 17/952,620, filed on Sep. 26, 2022, and entitled “AN APPARATUS FOR ENHANCING LONGEVITY AND METHOD FOR ITS USE,” the entirety of which is incorporated by reference herein in its entirety.

With continued reference to FIG. 1, in some embodiments, longevity decline driver 124 may be classified to longevity decline stage 128 using a lookup table. In an embodiment, the lookup table may relate longevity decline driver 124 and/or driver weight to longevity decline stage 128. As a non-limiting example, the lookup table may relate radiation exposure to a first stage. As another non-limiting example, processor 104 may “lookup” a given longevity decline driver 124 such as compromised autophagy in order to find a corresponding longevity decline stage 128, such as a second stage.

With continued reference to FIG. 1, in some embodiments, processor 104 may generate longevity plan 140 as a function of a longevity decline stage 128. As used in this disclosure, a “longevity plan” is a set of corrective measures configured to strategically improve longevity. In some embodiments, longevity plan 140 may be used to prevent decline of longevity. In some embodiments, the corrective measures may include a change in dietary habits, substance abuse, exercise habits, social habits, sleeping habits, and the like. Longevity plan 140 may include one or more modalities including but not limited to medical therapies, prescription medications, exercise, supplements, spirituality, and the like. For example, a longevity plan 140 may include the use of stem cell based therapies, anti-inflammatory drugs, blood-borne juvenile factors, elimination of damaged cells, telomerase reactivation, epigenetic drugs, activation of chaperones and protease systems, Mitohormetics, mitophagy, clearance of senescent cells, IIS and mTOR inhibition, AMPK and sirtins activation. The longevity plan 140 may be monitored in conjunction with one or more longevity parameters 112, longevity decline driver 124, or longevity decline stage 128 to provide feedback to a user and determine any adjustments and/or modifications. The information gathered from as a result of monitoring the effects of a longevity plan 140 may then be used in a feedback loop to determine a new longevity parameter 112, longevity decline driver 124 or longevity decline stage 128 based on how a user progresses through a particular program. The new longevity parameter 112, longevity decline driver 124 and/or longevity decline stage 128 may be used to generate a new longevity plan 140 or update the existing longevity plan 140.

With continued reference to FIG. 1, in some embodiments, longevity plan 140 for a first stage may include changes in lifestyle choices, controlled diet, avoidance of strong electric and magnetic fields, and similar corrective measures. As a non-limiting example, longevity plan 140 may include adequate sleep for the first stage of longevity decline stage 128 of sleep deprivation. As another non-limiting example, longevity plan 140 may include avoidance of strong electric and magnetic fields for the first stage of longevity decline stage 128 of strong exposure to electric and magnetic field.

With continued reference to FIG. 1, in some embodiments, longevity plan 140 for a second stage may include genetic repair, enhanced proteostasis, enhanced autophagy, reestablishment of gut function and microbiome balance, inflammation balancing/redox balancing, orchestration of the best environment and interventions to optimize epigenetics, optimal cycling of nutrient sensing, rebuilding the immune system, oncogenic potential mitigation, BHRT and T3/T4 optimization, telomerase, stem cell rejuvenation, seno-preventives, senomorphics, senolytics, mitochondrial preservation and mitophagy, restoration of altered intracellular communication, and similar corrective measures. As a non-limiting example, the longevity plan may include genetic repair for genomic instability in the second stage. As another non-limiting example, the longevity plan may include enhanced proteostasis for loss of proteostasis in the second stage. As another non-limiting example, the longevity plan may include enhanced autophagy for compromised autophagy in the second stage. As another non-limiting example, the longevity plan may include reestablishment of gut function and/or microbiome balance for microbiome disturbance in the second stage. As another non-limiting example, the longevity plan may include inflammation balancing/redox balancing for inflammation/oxidative stress in the second stage. As another non-limiting example, the longevity plan may include orchestration of the best environment and/or interventions to optimize epigenetics for epigenetic alterations in the second stage. As another non-limiting example, the longevity plan may include optical cycling of nutrient sensing for deregulated nutrient sensing (i.e. NAD/NADH SIRTs, GH/IGF-1/ATP and nutrient depletion mTOR/AMPK, Insulin/Glucose) in the second stage. As another non-limiting example, longevity plan 140 may include rebuilding the immune system for immuno-senescence in the second stage. As another non-limiting example, longevity plan 140 may include oncogenic potential mitigation for oncogenic potential in the second stage. As another non-limiting example, longevity plan 140 may include BHRT and/or T3/T4 optimization for hormone and thyroid depletion in the second stage. As another non-limiting example, longevity plan 140 may include telomerase for telomere attrition in the second stage. As another non-limiting example, longevity plan 140 may include stem cell rejuvenation for stem cell exhaustion in the second stage. As another non-limiting example, longevity plan 140 may include seno-preventives, senomorphics, and/or senolytics for cellular senescence and SASP in the second stage. As another non-limiting example, longevity plan 140 may include mitochondrial preservation and/or mitophagy for mitochondrial dysfunction in the second stage. As another non-limiting example, longevity plan 140 may include restoration of altered intracellular communication for altered intracellular communication in the second stage.

With continued reference to FIG. 1, processor 104 may be configured to generate a longevity plan 140 using a plan machine learning model 136. As used in this disclosure, a “plan machine learning model” is a machine learning model that generates a longevity plan. The plan machine learning model 136 disclosed herein may be consistent with a machine learning module disclosed with respect to FIG. 2. Inputs to the plan machine learning model 136 may include past or present longevity parameters 112, longevity decline stage 128, rate of aging, average longevity parameter, age comparison metric, user's anatomy data, longevity data, and the like. This data may be received from a database, such as longevity database 300. Previous longevity measurements 108, previous longevity parameter 112, and previous longevity decline stage 128, previous longevity plan 140 may come from the current user or users similarly situated to the users by health conditions, age, and fitness levels. Inputs into the plan machine learning model 136 may also include the output of the longevity classifier 132. The output of the plan machine learning model 136 may be a longevity plan 140 that is specific to the given user. The plan machine learning model 136 may be trained using longevity training data. Longevity training data is a plurality of data entries containing a plurality of inputs that are correlated to a plurality of outputs for training a processor by a machine-learning process to generate a longevity plan 140. Additionally, without limitation, longevity plan 140 disclosed herein may be consistent with a symphonic longevity plan in U.S. patent application Ser. No. 18/073,064, filed Dec. 1, 2022, and entitled “AN APPARATUS FOR ENHANCING LONGEVITY AND METHOD FOR ITS USE,” the entirety of which is incorporated by reference herein in its entirety.

With continued reference to FIG. 1, in some embodiments, longevity plan 140 may be generated using a lookup table. In an embodiment, a connection lookup table may relate longevity decline driver 124 and/or longevity decline stage 128 to longevity plan 140. As a non-limiting example, the lookup table may relate a first stage of EMF exposure to less exposure to EMF. As another non-limiting example, processor 104 may “lookup” a given longevity decline stage 128 such as stem cell exhaustion from a second stage in order to find a corresponding longevity plan 140, such as stem cell rejuvenation. In some embodiments, longevity plan 140 may be generated as a function of one or more machine-learning processes. In some embodiments, the machine-learning process may be trained with training data, wherein the training data may contain a plurality of inputs containing longevity decline stage 128 correlated to a plurality of outputs of longevity plan 140. As a non-limiting example the training data may include EMF exposure. As a non-limiting example, the plurality of outputs may include less exposure to EMF.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

With continued reference to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example inputs may include longevity measurement, and outputs may include target longevity factor. As another non-limiting example, inputs may include longevity treatment and corresponding longevity measurements, and outputs may include longevity treatment protocol.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to the target longevity factor, and the longevity treatment protocol.

With continued reference to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described above in this disclosure as inputs, outputs as described above in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With continued reference to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

With continued reference to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

With continued reference to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Referring now to FIG. 3, a non-limiting exemplary embodiment of a longevity database 300 is illustrated. In some embodiments, longevity database 300 may be consistent with longevity database 116. Processor 104 may be communicatively connected with longevity database 300. For example, in some cases, longevity database 300 may be local to processor 104. Alternatively, or additionally, in some cases, longevity database 300 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connects directly, dynamically, and non-hierarchically to as many other computing devices as possible. A “network topology” as used in this disclosure is an arrangement of elements of a communication network. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

With continued reference to FIG. 3, at least the processor 104 may, alternatively or additionally, store and/or retrieve data from, longevity parameter 112, longevity decline driver 124, longevity decline stage 128, and longevity plan 140. Determinations by a machine learning process may also be stored and/or retrieved from the longevity database 300, for instance in non-limiting examples a misreporting factor. As a non-limiting example, longevity database 300 may organize data according to one or more longevity database 300 tables. One or more database tables may be linked to one another by, for instance in a non-limiting example, common column values. For instance, a common column between two tables of database may include an identifier of a submission, such as a form entry, textual submission, research paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which data from one or more tables may be linked and/or related to data in one or more other tables.

With continued reference to FIG. 3, in a non-limiting embodiment, one or more longevity database tables of a database may include, but is not limited to, a longevity measurement, which may include longevity measurement for use in identifying longevity measurement of a user and/or correlating longevity marker data, entries indicating values and/or degrees of relevance to and/or efficacy in identifying longevity measurement pertaining to a user, and/or other elements of processor 104 and/or apparatus 100 may use to determine values and/or usefulness and/or relevance of longevity marker data in identifying longevity measurements as described in this disclosure. One or more tables may include a longevity measurement, which may correlate longevity markers and/or combinations thereof to one or more longevity measurements; longevity measurement may contain a plurality of entries associating at least an element of longevity marker with longevity measurement. One or more tables may include, without limitation, a longevity parameter 112, which may contain one or more inputs identifying one or more categories of data, for instance demographic data, medical history data, physiological data, or the like. One or more tables may include a longevity parameter 112. One or more tables may include, without limitation, a longevity decline driver 124. In some embodiments, longevity parameter 308 may include driver weight. One or more tables may include, without limitation, a longevity decline stage 128, which may include first stage, second stage, third stage, and the like. One or more tables may include, without limitation, a longevity plan 140, which may include one or more entries indicating the longevity treatment corresponding to one or more longevity stage.

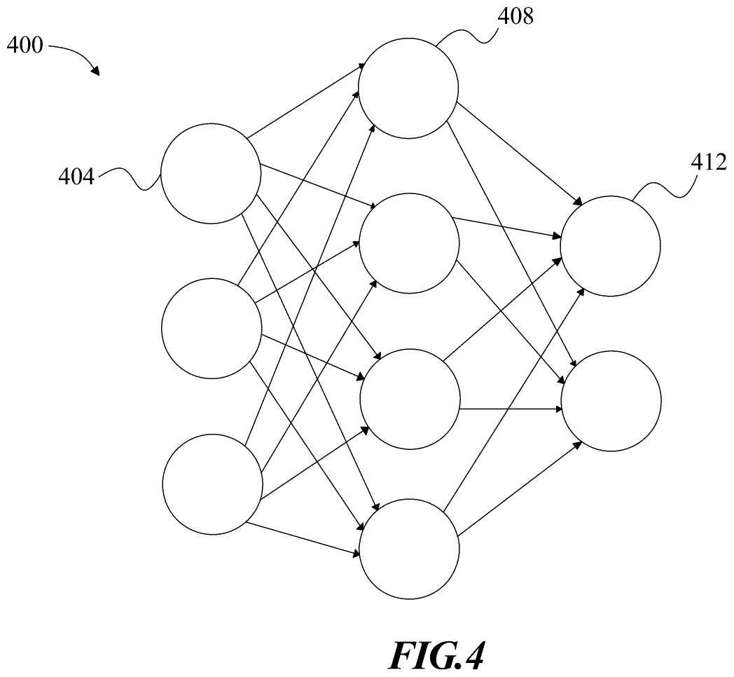

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

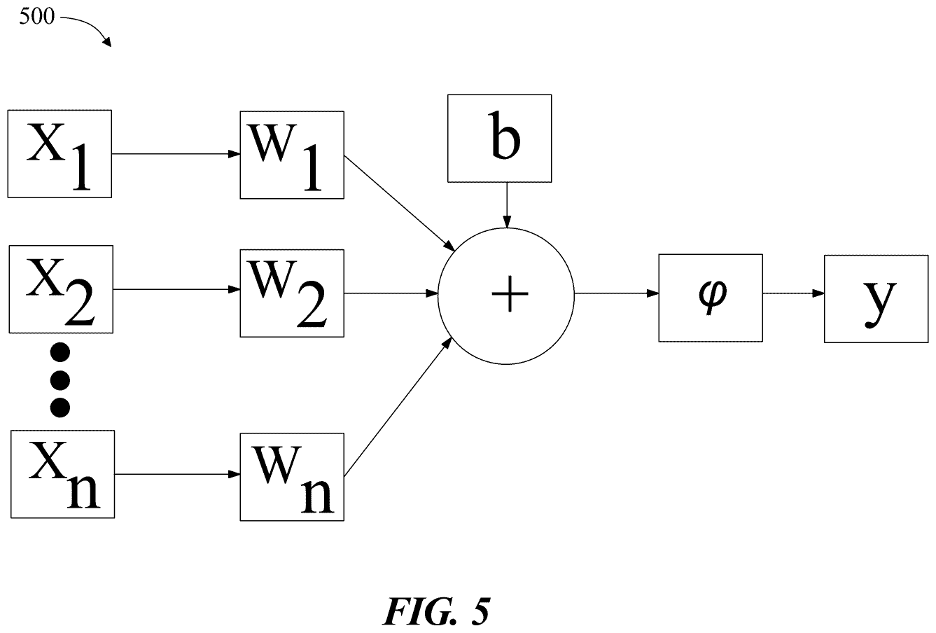

Referring now to FIG. 5, an exemplary embodiment of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wi may be determined by training a neural network using training data, which may be performed using any suitable process as described above.

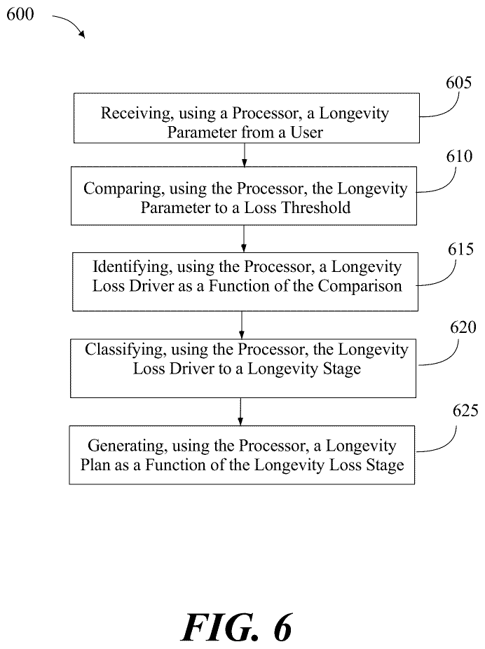

Referring now to FIG. 6, an exemplary method 600 for preventing loss of longevity is illustrated. Method 600 may include a step 605 of receiving, using a processor, a longevity parameter from a user. Method may include a step 610 of comparing, using the processor, the longevity parameter to a decline threshold. In some embodiments, the decline threshold may include a historical longevity parameter. In some embodiments, the processor may further include generating, using a threshold machine-learning model, the decline threshold. This may be implemented as disclosed with reference to FIGS. 1-5.

With continued reference to FIG. 6, in some embodiments, method 600 may include a step 615 of identifying, using the processor, a longevity decline driver as a function of a comparison. In some embodiments, the longevity decline driver may include a driver weight. In some embodiments, identifying the longevity decline driver may include identifying a plurality of longevity decline drivers and determining a core longevity decline driver within the plurality of longevity decline driver. In some embodiments, the core longevity decline driver may include the longevity decline driver with a maximum driver weight. This may be implemented as disclosed with reference to FIGS. 1-5.

With continued reference to FIG. 6, in some embodiments, method 600 may include a step 620 of classifying, using the processor, the longevity decline driver to a longevity decline stage. In some embodiments, the longevity decline stage may include a first stage, wherein the first stage may include a stage where loss of longevity is self-preventable. In some embodiments, the longevity decline stage may include a second stage, wherein the second stage may include a stage where loss of longevity requires professional intervention. In some embodiments, the longevity decline stage may include a third stage, wherein the third stage comprises a stage wherein an ability of the user to return to a health state is precluded. In some embodiments, the processor may further include classifying, using a longevity classifier, the longevity decline driver to the longevity decline stage. This may be implemented as disclosed with reference to FIGS. 1-5.

With continued reference to FIG. 6, in some embodiments, method 600 may include a step 625 of generating, using the processor, a longevity plan as a function of the longevity decline stage. This may be implemented as disclosed with reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

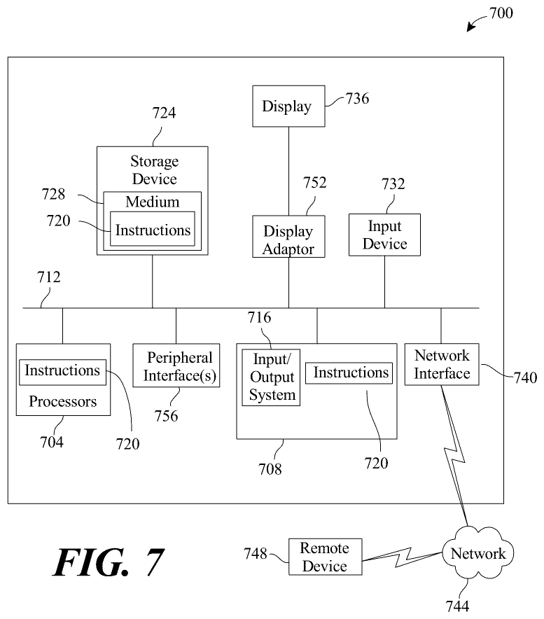

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for preventing loss of longevity, wherein the apparatus comprises:
at least a processor; and
a memory communicatively connected to the at least a processor, the memory containing instructions configuring the at least a processor to:
receive a longevity parameter of a user from a wearable device comprising a biosensor and a user interface configured to receive input from the user, the input comprising information associated with a longevity of the user;
generate a decline threshold by:
selecting training data comprising a plurality longevity parameters correlated to a plurality of decline thresholds, wherein the correlations are derived from a data structure comprising an array indexing operation configured maps input values to output values in order to optimize a runtime of the processor;
training a threshold machine-learning model using the training data; and
outputting the decline threshold;
compare the longevity parameter to the decline threshold;
identify a longevity decline driver as a function of the comparison, wherein identifying the longevity decline driver comprises:
generating a driver machine learning model;
receiving driver training data comprising a plurality of comparisons of longevity parameters and decline thresholds correlated to longevity decline drivers, wherein receiving the driver training data comprises processing the training data using a training data classifier;
training the driver machine learning model as a function of the processed driver training data, wherein the driver training data comprises previous outputs from the driver machine learning model; and
determining the longevity decline driver using the trained driver machine learning model;
classify the longevity decline driver to a longevity decline stage; and
generate a longevity plan as a function of the longevity decline stage using a lookup table comprising relationships between a plurality of longevity decline stages and a plurality of longevity plans.

2. The apparatus of claim 1, wherein the longevity decline driver comprises a driver weight.

3. The apparatus of claim 1, wherein identifying the longevity decline driver comprises:
identifying a plurality of longevity decline drivers; and
determining a core longevity decline driver within the plurality of longevity decline drivers.

4. The apparatus of claim 3, wherein the core longevity decline driver comprises the longevity decline driver with a maximum driver weight.

5. The apparatus of claim 1, wherein the longevity decline stage comprises a first stage, wherein the first stage comprises a stage where loss of longevity is self-preventable.

6. The apparatus of claim 1, wherein the longevity decline stage comprises a second stage, wherein the second stage comprises a stage where loss of longevity requires professional intervention.

7. The apparatus of claim 1, wherein the longevity decline stage comprises a third stage, wherein the third stage comprises a stage wherein an ability of the user to return to a health state is precluded.

8. The apparatus of claim 1, wherein the decline threshold comprises a historical longevity parameter.

9. The apparatus of claim 1, wherein the memory contains instructions configuring at least the processor to generate the decline threshold using a threshold machine-learning model.

10. The apparatus of claim 1, wherein the memory contains instructions configuring at least the processor to classify the longevity decline driver to the longevity decline stage using a longevity classifier.

11. A method for preventing loss of longevity, wherein the method comprises:
receiving, using a processor, a longevity parameter of a user from a wearable device comprising a biosensor and a user interface configured to receive input from the user, the input comprising information associated with a longevity of the user;
generating, using the processor, a decline threshold by:
selecting training data comprising a plurality longevity parameters correlated to a plurality of decline thresholds, wherein the correlations are derived from a data structure comprising an array indexing operation configured maps input values to output values in order to optimize a runtime of the processor;
training a threshold machine-learning model using the training data; and
outputting the decline threshold;
comparing, using the processor, the longevity parameter to the decline threshold;
identifying, using the processor, a longevity decline driver as a function of the comparison, wherein identifying the longevity decline driver comprises:
generating a driver machine learning model;
receiving driver training data comprising a plurality of comparisons of longevity parameters and decline thresholds correlated to longevity decline drivers, wherein receiving the driver training data comprises processing the training data using a training data classifier;
training the driver machine learning model as a function of the processed driver training data, wherein the driver training data further comprises previous outputs from the driver machine learning model; and
determining the longevity decline driver using the trained driver machine learning model;
classifying, using the processor, the longevity decline driver to a longevity decline stage; and
generating, using the processor, a longevity plan as a function of the longevity decline stage using a lookup table comprising relationships between a plurality of longevity decline stages and a plurality of longevity plans.

12. The method of claim 11, wherein the longevity decline driver comprises a driver weight.

13. The method of claim 11, wherein identifying the longevity decline driver comprises:
identifying a plurality of longevity decline drivers; and
determining a core longevity decline driver within the plurality of longevity decline driver.

14. The method of claim 13, wherein the core longevity decline driver comprises the longevity decline driver with a maximum driver weight.

15. The method of claim 11, wherein the longevity decline stage comprises a first stage, wherein the first stage comprises a stage where loss of longevity is self-preventable.

16. The method of claim 11, wherein the longevity decline stage comprises a second stage, wherein the second stage comprises a stage where loss of longevity requires professional intervention.

17. The method of claim 11, wherein the longevity decline stage comprises a third stage, wherein the third stage comprises a stage wherein an ability of the user to return to a health state is precluded.

18. The method of claim 11, wherein the decline threshold comprises a historical longevity parameter.

19. The method of claim 11, further comprising:
generating, using a threshold machine-learning model, the decline threshold.

20. The method of claim 11, further comprising:
classifying, using a longevity classifier, the longevity decline driver to the longevity decline stage.
